(19) United States
(12) Patent Application Publication
(10) Pub. No.: US 2023/0193264 A1
(43) Pub. Date: Jun. 22, 2023

(54) METHODS AND COMPOSITIONS FOR IMPROVED TYPE I-E CRISPR BASED GENE SILENCING

(71) Applicant: Duke University, Durham, NC (US)

(72) Inventors: Michael D. Lynch, Durham, NC (US); Zhixia Ye, Raleigh, NC (US); Eirik Moreb, Durham, NC (US); Juliana Lebeau, Durham, NC (US)

(21) Appl. No.: 17/906,372

(22) PCT Filed: Mar. 16, 2021
(86) PCT No.: PCT/US21/22583

§ 371 Date: Sep. 15, 2022

Related U.S. Application Data
(60) Provisional application No. 62/990,172, filed on Mar. 16, 2020.

(51) Int. Cl. C12N 15/113; C12N 15/52; C12N 9/22

(52) U.S. Cl. CPC C12N 15/113; C12N 15/52; C12N 9/22; C12N 2310/20

(57) ABSTRACT

CRISPR based interference has become common in various application form genetic circuits to dynamic metabolic control. In E. coli, the native CRISPR Cascade system can be utilized for silencing by deletion of the cas3 nuclease along with expression of guide RNA arrays, where multiple genes can be silenced from a single transcript.

### CROSS-REFERENCE TO RELATED APPLICATIONS

This application claims priority to U.S. Provisional Patent Application No. 62/990,172 filed Mar. 16, 2020, which is incorporated by reference herein in its entirety.

### STATEMENT AS TO FEDERALLY SPONSORED RESEARCH

This invention was made with government support under 12043956 awarded by Office of Naval Research; and under EE0007563 awarded by DOE EERE. The government has certain rights in the invention.

### REFERENCE TO A SEQUENCE LISTING

The instant application contains a Sequence Listing which has been filed electronically in ASCII format as 49186-45_ST25.txt created on Mar. 10, 2020, which is 28943 bytes in size, and is hereby incorporated by reference in its entirety.

### BACKGROUND

Gene silencing is a powerful tool and CRISPR based methods have increased the simplicity of this approach (Adli, M. The CRISPR tool kit for genome editing and beyond. Nat. Commun. 9, 1911 (2018). In E. coli, the native multi-protein Cascade (type I-E CRISPR) system can be engineered for use in gene silencing, which involved deletion of the nuclease component and overexpression of the genes responsible processing CRISPR arrays and target DNA binding. One benefit of using the modified Cascade system is the targeting of multiple genes with the expression of a single transcript containing multiple protospacers, which is subsequently processed into individual guide RNAs (Luo, M. L., Mullis, A. S., Leenay, R. T. & Beisel, C. L. Repurposing endogenous type I CRISPR-Cas systems for programmable gene repression. Nucleic Acids Research vol. 43 674-681 (2015)).

### SUMMARY

CRISPR based interference has become common in various applications from genetic circuits to dynamic metabolic control. Cas 1/2 endonuclease mediated guide array instability has been identified as an issue in some cases. In E. coli the native CRISPR Cascade system can be utilized for silencing by deletion of the cas3 nuclease along with expression of guide arrays, where multiple genes can be silenced from a single transcript.

### BRIEF DESCRIPTION OF DRAWINGS

FIG. 1(A)-1(G): FIG. 1(A) a guide array schematic. FIG. 1(B) an example of guide array protospacer loss. FIG. 1(C) protospacer modification as quantified by PCR. FIG. 1(D) guide array stability as a function of guide array and host strain. FIG. 1(E) schematic for complementation of fabI silencing with pFABI. FIG. 1(F) graph demonstrating colony counts and FIG. 1(G) demonstrating guide array stability with strains transformed with guide arrays and pFABI.

FIG. 2 represents an exemplary collection of plasmids of the invention.

FIG. 3 represents exemplary strains of the invention.

FIG. 4 represents a summary of exemplary sgRNA guide sequences and primers for their construction. Spacers are italicized.

FIG. 5 represents a summary of exemplary synthetic DNA of the invention.

### DETAILED DESCRIPTION

### General Definitions

As used in the specification and the claims, the singular forms “a,” “an,” and “the” include plural referents unless the context clearly dictates otherwise. Thus, for example, reference to an “expression vector” includes a single expression vector as well as a plurality of expression vectors, either the same (e.g., the same operon) or different; reference to “microorganism” includes a single microorganism as well as a plurality of microorganisms; and the like.

The term “heterologous DNA,” “heterologous nucleic acid sequence,” and the like as used herein refers to a nucleic acid sequence wherein at least one of the following is true: (a) the sequence of nucleic acids is foreign to (i.e., not naturally found in) a given host microorganism; (b) the sequence may be naturally found in a given host microorganism, but in an unnatural (e.g., greater than expected) amount; or (c) the sequence of nucleic acids comprises two or more subsequences that are not found in the same relationship to each other in nature. For example, regarding instance (c), a heterologous nucleic acid sequence that is recombinantly produced will have two or more sequences from unrelated genes arranged to make a new functional nucleic acid, such as a nonnative promoter driving gene expression.

Species and other phylogenic identifications are according to the classification known to a person skilled in the art of microbiology.

Enzymes are listed here within, with reference to a UniProt identification number, which would be well known to one skilled in the art. The UniProt database can be accessed at http://www.UniProt.org/. When the genetic modification of a gene product, i.e., an enzyme, is referred to herein, including the claims, it is understood that the genetic modification is of a nucleic acid sequence, such as or including the gene, that normally encodes the stated gene product, i.e., the enzyme.

Where methods and steps described herein indicate certain events occurring in certain order, those of ordinary skill in the art will recognize that the ordering of certain steps may be modified and that such modifications are in accordance with the variations of the invention. Additionally, certain steps may be performed concurrently in a parallel process when possible, as well as performed sequentially.

The meaning of abbreviations is as follows: “C” means Celsius or degrees Celsius, as is clear from its usage, DCW means dry cell weight, “s” means second(s), “min” means minute(s), “h,” “hr,” or “hrs” means hour(s), “psi” means pounds per square inch, “nm” means nanometers, “d” means day(s), “μL” or “uL” or “ul” means microliter(s), “mL” means milliliter(s), “L” means liter(s), “mm” means millimeter(s), “nm” means nanometers, “mM” means millimolar, “μM” or “uM” means micromolar, “M” means molar, “mmol” means millimole(s), “μmol” or “uMol” means micromole(s)”, “g” means gram(s), “μg” or “ug” means microgram(s) and “ng” means nanogram(s), “PCR” means polymerase chain reaction, “OD” means optical density, “OD600” means the optical density measured at a photon wavelength of 600 nm, “kDa” means kilodaltons, “g” means the gravitation constant, “bp” means base pair(s), “kbp” means kilobase pair(s), “% w/v” means weight/volume percent, “% v/v” means volume/volume percent, “IPTG” means isopropyl-μ-D-thiogalactopyranoiside, “aTc” means anhydrotetracycline, “RBS” means ribosome binding site, “rpm” means revolutions per minute, “HPLC” means high performance liquid chromatography, and “GC” means gas chromatography.

Unless otherwise defined, all technical terms used herein have the same meaning as commonly understood by one of ordinary skill in the art to which this disclosure belongs.

### Microorganisms

Features as described and claimed herein may be provided in a microorganism selected from the listing herein, or another suitable microorganism, that also comprises one or more natural, introduced, or enhanced product bio-production pathways. Thus, in some embodiments the microorganism(s) comprise an endogenous product production pathway (which may, in some such embodiments, be enhanced), whereas in other embodiments the microorganism does not comprise an endogenous product production pathway.

More particularly, based on the various criteria described herein, suitable microbial hosts for the bio-production of a chemical product generally may include, but are not limited to the organisms described in the Methods Section.

The host microorganism or the source microorganism for any gene or protein described here may be selected from the following list of microorgansims: Citrobacter, Enterobacter, Clostridium, Klebsiella, Aerobacter, Lactobacillus, Aspergillus, Saccharomyces, Schizosaccharomyces, Zygosaccharomyces, Pichia, Kluyveromyces, Candida, Hansenula, Debaryomyces, Mucor, Torulopsis, Methylobacter, Escherichia, Salmonella, Bacillus, Streptomyces, and Pseudomonas. In some aspects the host microorganism is an E.coli microorganism.

### Overview of Invention Aspects

In general, unstable guide arrays may be eliminated by the use of a genetically modified microorganism characterized by an endogenous cas3 nuclease that is deleted or mutated; a Cascade operon may be overexpressed; and at least one CRISPR/Cascade gRNA maybe expressed to result in reduced expression of at least one gene.

In some aspects, the microorganism and any method using the microorganism may comprise the use of a genetically modified microorganism having a deletion or mutation of an endogenous cas1 gene.

In some aspects, the microorganism and any method using the microorganism may include the use of a genetically modified microorganism is further characterized by the Cascade operon under the control of an inducible promoter that is a PhoB activated.

In some aspects, the microorganism and any method using the microorganism may include the use of a genetically modified microorganism that is an E. coli microorganism.

In some aspects, the microorganism and any method using the microorganism may function to reduce expression of a gene that is: fabI, gltA1, gltA2, udhA, zwf, or a combination thereof.

### Detailed Description

Unstable guide arrays may be due to expression of the Cas 1/2 endonuclease complex. cas1 deletion reduces guide array instability. Basal Cas 1/2 endonuclease activity results in the loss of protospacers from guide arrays. Subsequently, guide arrays may become ineffective in silencing can be amplified through selection. Replacing a constitutive promoter driving Cascade complex expression with a tightly controlled inducible promoter improves guide array stability.

Unstable guide arrays are also eliminated when a method of conditionally silencing a gene in a genetically modified microorganism, including providing a genetically modified microorganism characterized by deletion or mutation of an endogenous cas3 nuclease; a Cascade operon; and at least one CRISPR/Cascade gRNA. The method including the step of growing the genetically modified microorganism under conditions wherein expression of the CRISPR/Cascade gRNA results in reduced expression of at least one gene of the genetically modified microorganism. The microorgansims and methods of using these microorganisms of the invention may include any combination of deletion or selective mutation of the endogenous cas3 nuclease gene, or conditional expression of a Cascade operon. One or both of these conditions result in increased stability of the guide array.

The guide array may include a single gRNA that results in increased transcriptional silencing of a single gene upon the conditional expression of the array. Alternatively, the guide array may include more than one gRNA resulting in transcriptional silencing of more than one gene. A single guide array may in include means to regulate one, two, three, four, five or more genes simultaneously. Alternatively, the genetically modified microorganism may contain two or more guide arrays simultaneously each of which may be conditionally expressed and will result in transcriptional silencing of one or more genes.

In one aspect, the method may comprise the use of a genetically modified microorganism having a deletion or mutation of an endogenous cas1 gene. The deletion or mutation of the cas1 gene may be combined with both conditions to provide optimal guide array stability—that is combined with both the deletion or selective mutation of the endogenous cas3 nuclease gene, or conditional expression of a Cascade operon. It is appreciated, however, that any combination of these three factors (cas3 deletion/mutation; cas1 deletion/mutation; or conditional Cascade operon expression), will in fact increase the stability of guide arrays.

Deletion or mutation of the cas3 and/or cas1 endogenous genes merely refers to any modification of the endogenous gene rendering expression of this endogenous gene impossible. The deletion or mutation may occur in gene regulatory sequences, or modification of the coding sequence of the gene itself, or other means of preventing expression of a specific endogenous gene of the genetically modified microorganism.

The phrase conditionally expressed, conditionally overexpressed, inducible promotor, or tightly repressed inducible promotor refer to means of regulating gene expression. Gene expression may be regulated conditionally by introduction of a stimulus or alternatively the withdrawal of required nutrient or other substance. A tightly repressed promotor sequence refers to the fact that regulation of gene expression strictly does not occur while promotor is under the described repressive conditions and inducible refer to the fact that a promotor may be responsive to an externally applied signal.

A guide array refers to any configuration permitting expression of gRNA specific for a target. In this case that target is a gene to be transcriptionally silenced under specific conditions.

Another aspect of the invention is described by comparison of guide array expression in genetically modified microorganisms having any combination of deletion or selective mutation of the endogenous cas3 nuclease gene, or conditional expression of a Cascade operon in contrast to genetically modified microorganism lacking these characteristics. These characteristics serve to enhance guide array stability and thus enhance transcriptional gene silencing of a target gene.

In one aspect, the method my comprise the use of a genetically modified microorganism is further characterized by the Cascade operon under the control of an inducible promoter that is a PhoB activated. It is appreciated that any inducible promotor other than PhoB is encompassed by the invention.

In one aspect, the method may comprise the use of a genetically modified microorganism that is an E. coli microorganism. It is appreciated however, that the genes to be regulated, deleted, or mutated as well as the operon and guide array to be expressed are applicable to any known microorganism.

In one aspect, the method may function to reduce expression of a gene that is: fabI, gltA1, gltA2, udhA, zwf, or a combination thereof. It is appreciated that while these genes have been identified as candidates for gene regulation in the genetically modified microorganism described herein, the methods and microorganism are widely applicable to any gene identified as desirious to selectively regulated. For example,

### EXAMPLES

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

### Materials and Methods

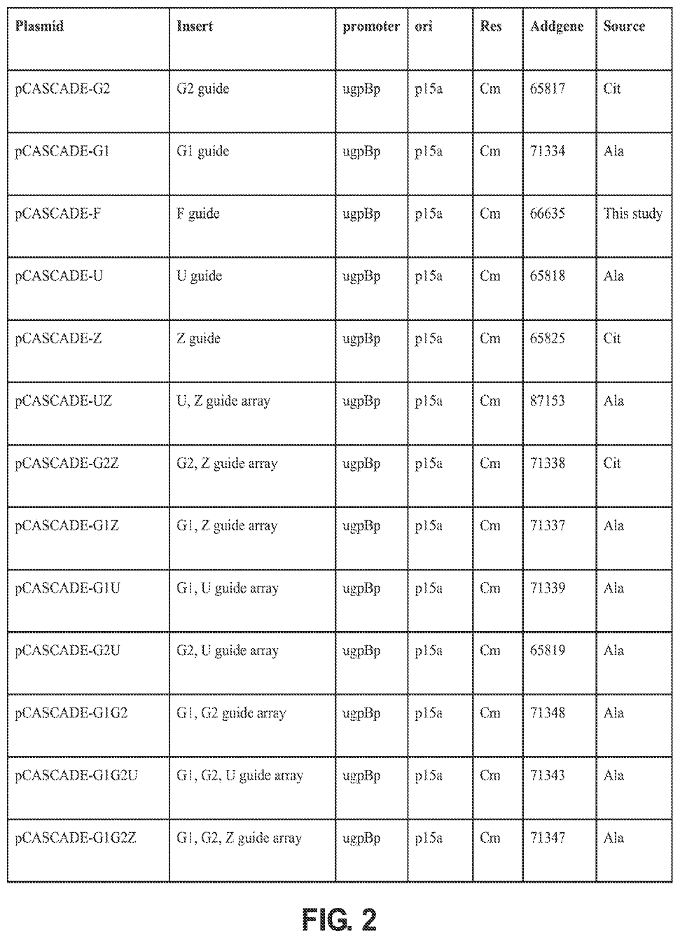

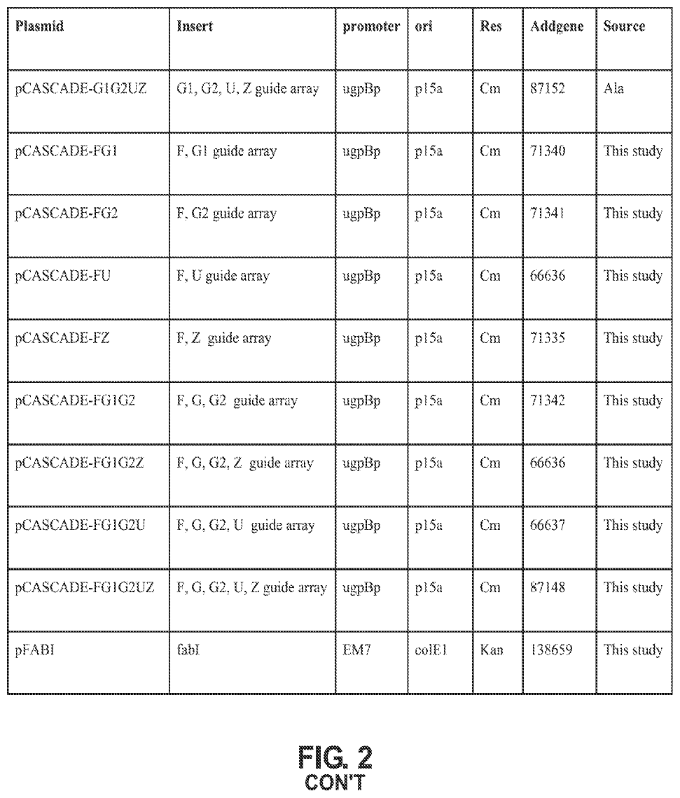

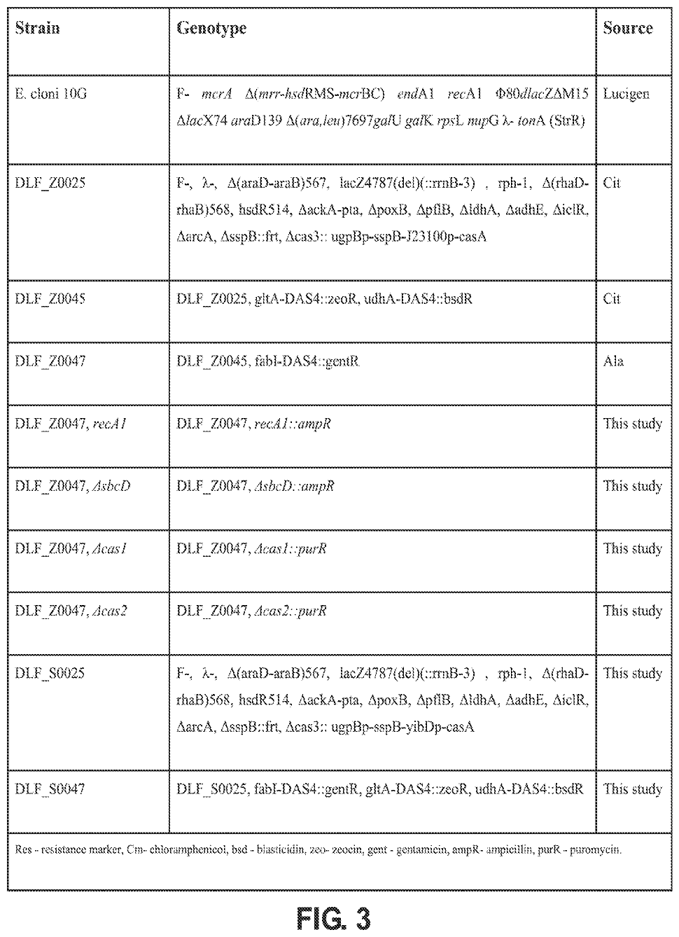

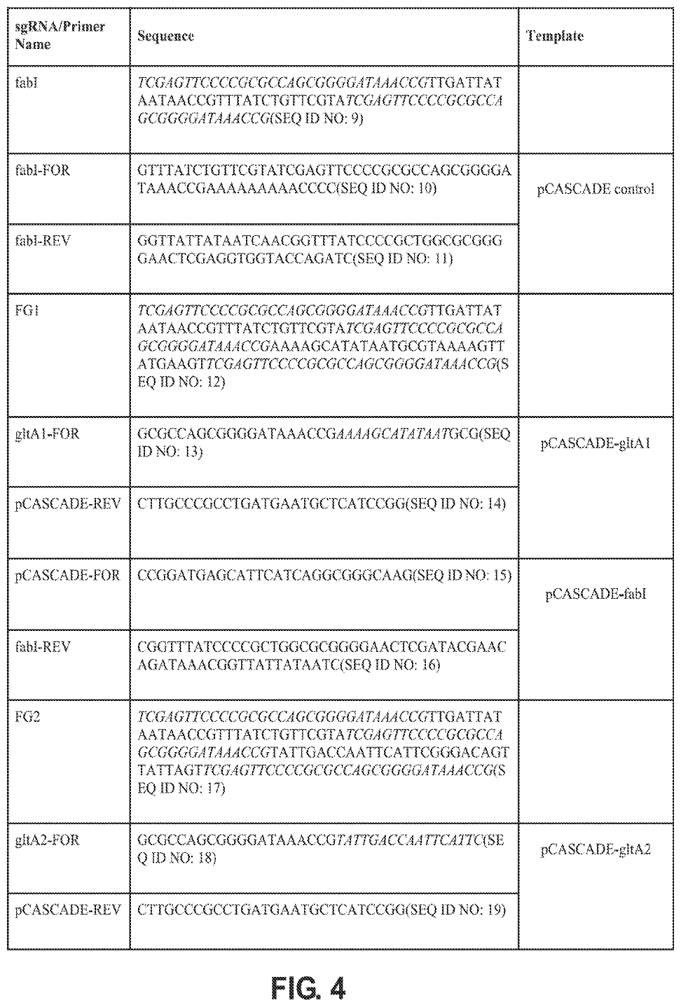

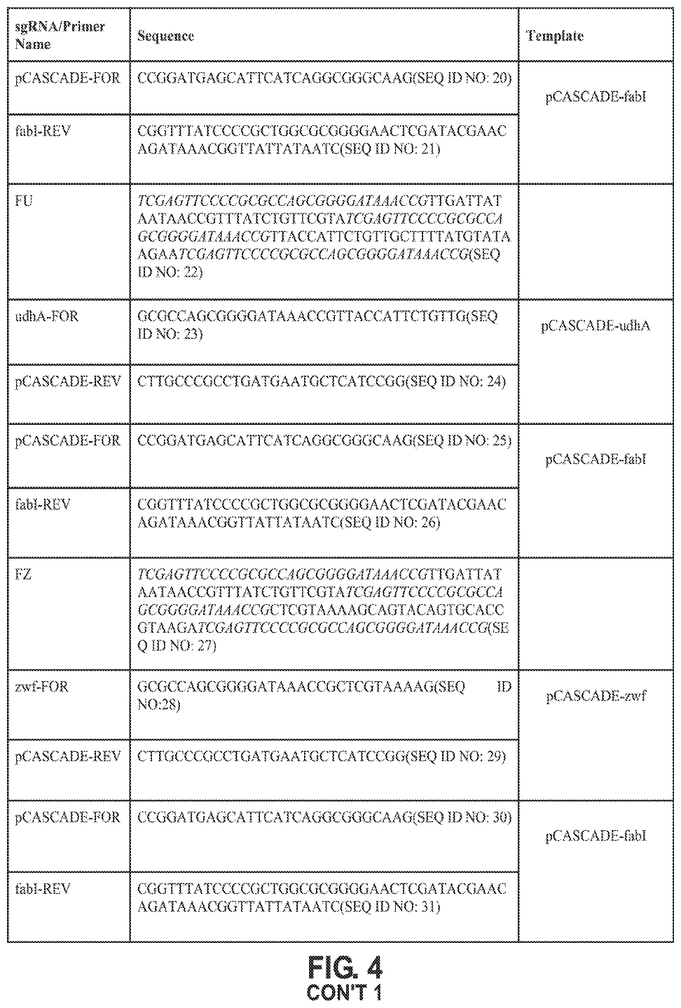

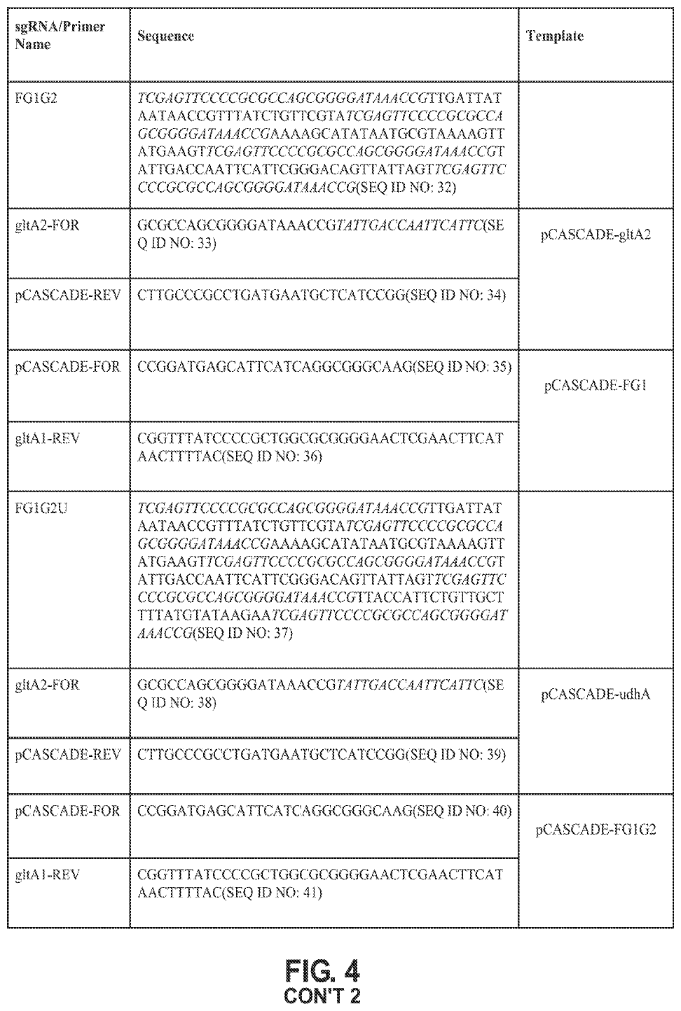

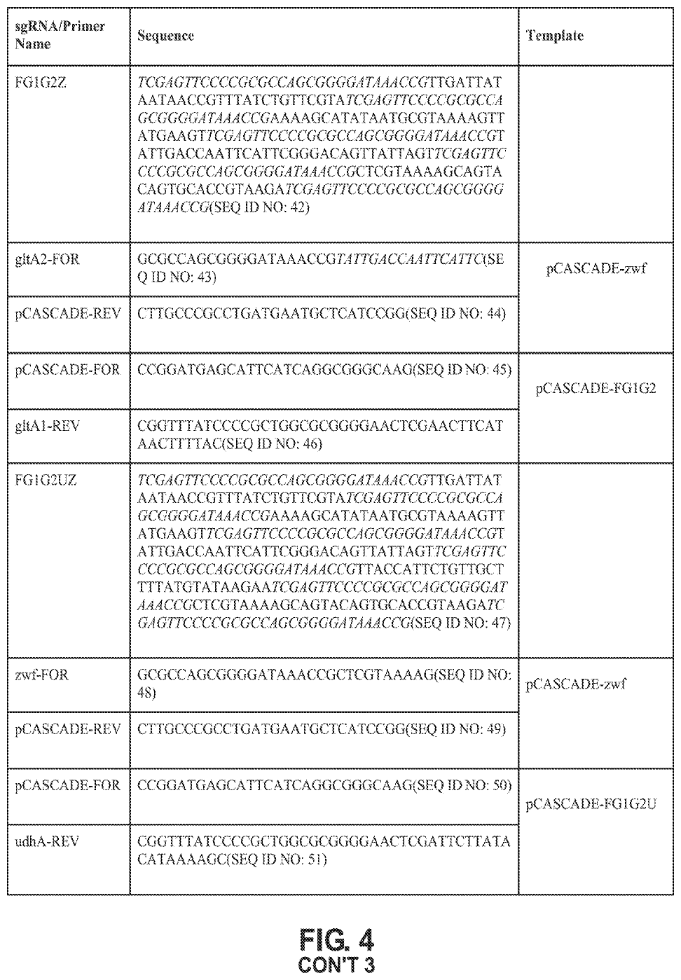

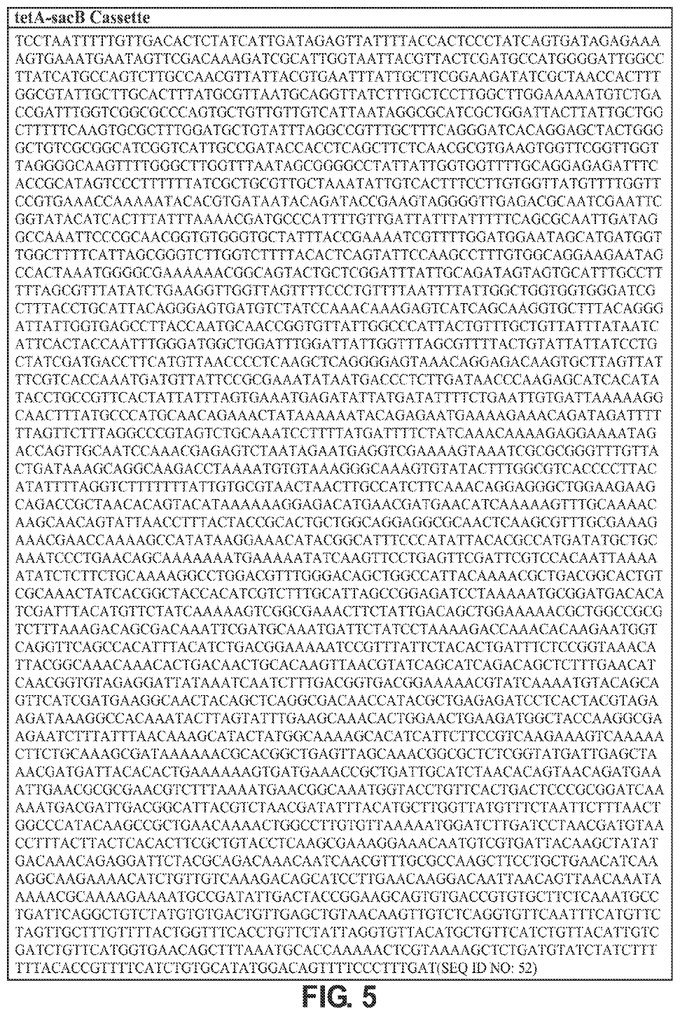

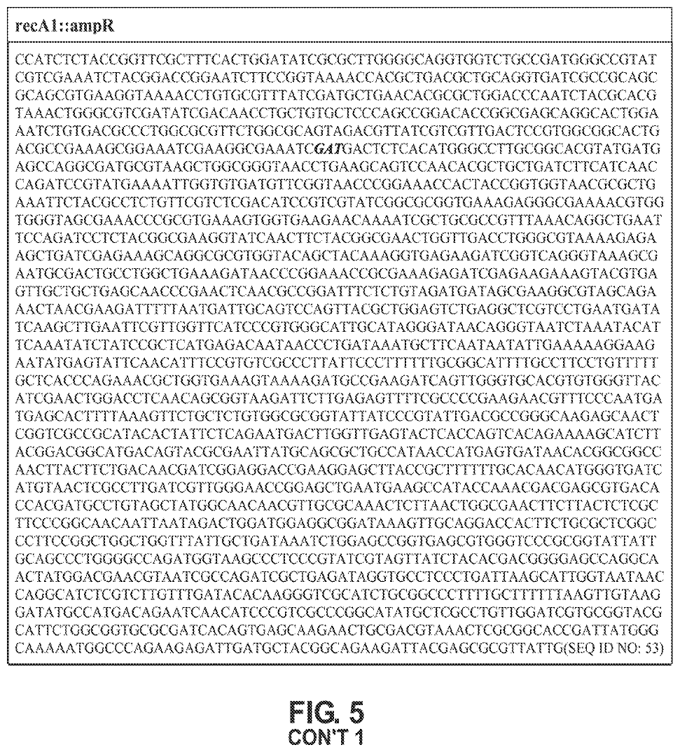

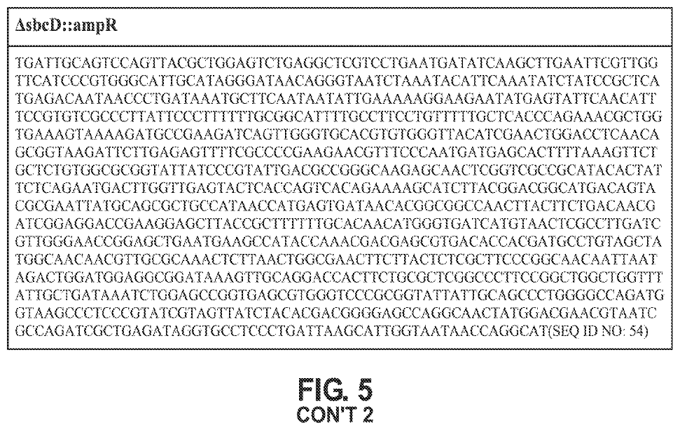

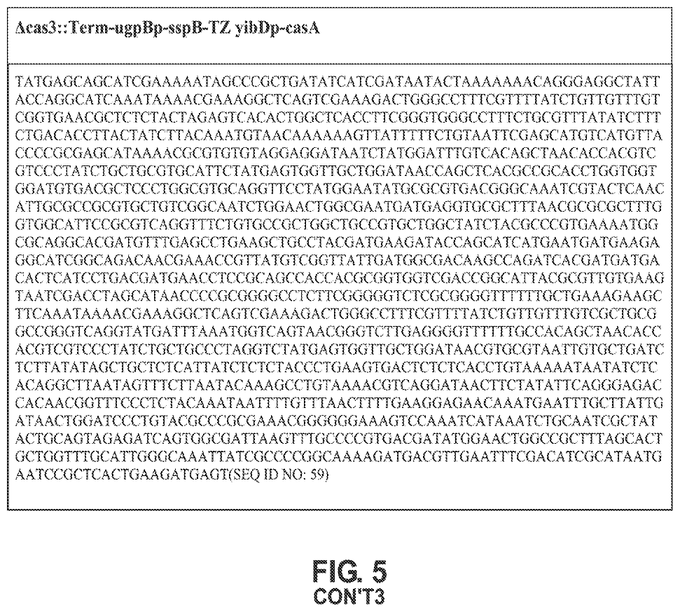

FIG. 2 summarizes exemplary plasmids of the invention. FIG. 3 summarizes exemplary microorganism strains of the invention are summarized. FIG. 4 summarizes a list of exemplary sgRNA guide sequences and primers used to construct them. FIG. 5 summarizes exemplary synthetic DNA of the invention. Spacers are italicized.

Reagents and Media: Unless otherwise stated, all materials and reagents were of the highest grade possible and purchased from Sigma (St. Louis, Mo.). Luria Broth, lennox formulation with lower salt was used for routine strain and plasmid propagation, construction, and colony isolation. Chloramphenicol, ampicillin, and tetracycline were used at a final working concentration of 20 μg/mL, 100 μg/mL, and 5 μg/mL respectively. Puromycin selection was performed using a final working concentration of 200 μg/mL, with LB supplemented with 50 mM potassium phosphate buffer (pH=8.0) to maintain pH for adequate selection.

Strains and Plasmids: pCASCADE array plasmids were constructed as previously reported using PCR assembly of smaller arrays. For pCASCADE plasmids constructed in this study, refer to FIGS. 2-5 for sequence and primer details. Plasmid, pFABI, was constructed to enable constitutive expression from a codon optimized fabI gene using the strong synthetic EM7 promoter. Plasmid DNA containing the promoter and gene was obtained from Twist Biosciences (San Francisco, Calif.). Strain E. cloni 10G was obtained from Lucigen. Strains DLF_Z0025, DLF_Z0045 and DLF_Z0047 were made as previously reported. All strains made in this study were constructed using standard recombineering. The recombineering plasmid pSIM5 and the tet-sacB selection/counterselection marker cassette were kind gifts from Donald Court (NCI, https://redrecombineering.ncifcrfgov/court-lab.html). DLF_Z0047 ΔsbcD::ampR was constructed via direct integration and gene replacement with linear donor DNA containing the appropriate antibiotic marker. The donor was prepared by PCR of synthetic ampicillin resistance cassette (ampR2) with primer del_sbcD_p1 and del_sbcD_p2. DLF_Z0047, recA1::ampR was similarly constructed, however the integration incorporated a G160D mutation into the recA gene rather than a deletion. Strains DLF_Z0047 Δcas1::purR and DLF_Z0047 Δcas2::purR were constructed via direct integration and gene replacement with linear donor DNA. Strains DLF_S0047 and DLF_S0025 were constructed from DLF_Z0047 and DLF_Z0025 respectively, using recombineering and tet-sacB based selection counterselection to replace the sspB gene and promoter in front of the Cascade operon. All genetic modifications were confirmed by PCR and sequencing. Sequencing was performed by either Genewiz (Morrisville, N.C.) or Eurofins (Louisville, Ky.). Plasmid transformations were accomplished using standard methods.

Guide Stability Testing: Plasmid DNA minipreps and sequencing were performed with standard methods. The following two primers were used to amplify guide arrays from pCASCADE plasmids gRNA-for: 5′-GGGAGACCACAACGG-3′(SEQ ID NO: 60), gRNA-rev: 5′-CGCAGTCGAACGACCG-3′(SEQ ID NO: 61). Colony PCR was performed as follows: 2X EconoTaq Master mix (Lucigen) was used in 10 μL PCR reactions consisting of 5 μL of 2X EconoTaq Master mix (Lucigen), 1 μL of each primer (10 uM concentration), 3 μL dH₂O and a small part of a colony. PCR parameters were an initial 98° C., 2 minute initial denaturation followed by 35 cycles of 94° C., 30 seconds, 60° C. 30 seconds, and 72° C., 30 seconds and a final 72° C., 5 min final extension. PCR products were then analyzed via agarose gel electrophoresis.

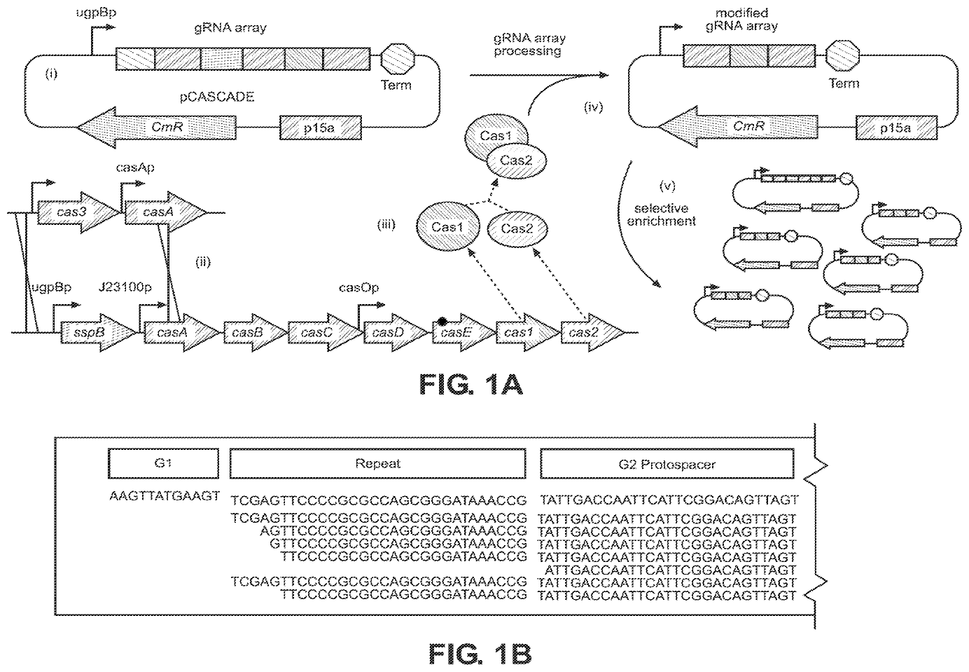

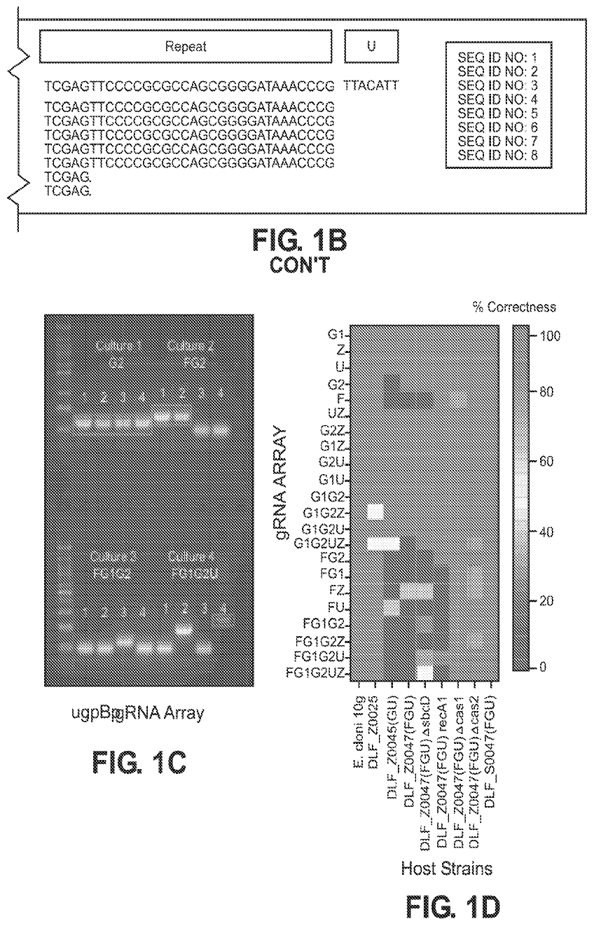

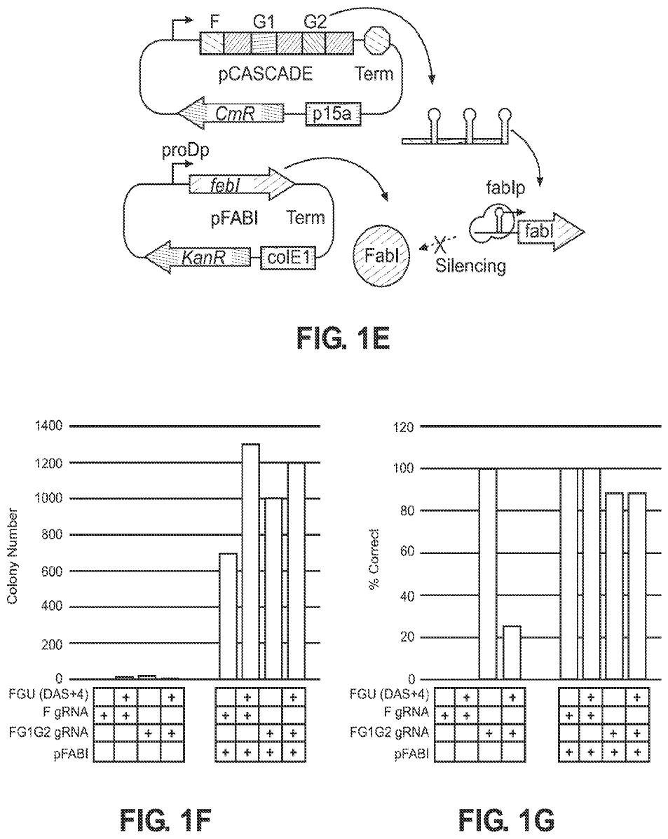

Referring now to FIG. 1A-B, first several guide array plasmids where guide loss was suspected were sequenced. As an example (FIG. 1A), we transformed a guide array plasmid containing protospacers to silence the gltAp1 (G1), gltAp2 (G2) and udhA (U) promoters, into a host strain (DLF_Z0047) engineered with degron tags capable of proteolytic degradation of FabI (enoyl-ACP reductase), GltA (citrate synthase) and UdhA (soluble transhydrogenase). A single colony was chosen and used to inoculate a 5 mL culture (Luria broth), and after overnight growth the culture was plated to isolate single colonies, 24 clones were isolated, and the guide array plasmid was miniprepped and sequenced. While 17 plasmids had the expected sequence and retained all 3 protospacers (FIG. 1B, top sequence), the other 7 had mutations, with loss of the 2 protospacers (G1 and U) flanking the middle protospacer G2. Four of these modified clones retained both the 5′ and 3′ flanking repeat sequences, whereas the other three also lost either the 5′ or 3′ repeat sequence flanking the G2 protospacer.

As shown in FIG. 1C, as a next step the stability of a single guide (G2) and three additional guide arrays: FG2, FG1G2 and FG1G2U, where “F” is a protospacer targeting the fabI promoter were evaluated. Again, using strain DLF_Z0047 and again starting with a single colony used to inoculate a 5 mL culture (Luria broth). After overnight growth the culture was plated to isolate single colonies. In this case, four clones from each of four cultures were isolated and colony PCR rather than sequencing was utilized to evaluate guide array stability. Results are given in FIG. 1C. While in this case the single G2 guide proved stable, the larger arrays of 2-4 protospacers had varied degrees of instability, producing amplicons consistent with the loss of 1-3 protospacers.

Referring now to FIG. 1D, with the success of PCR as a tool to assess stability, the next step of evaluation of guide array stability for a larger grouping of guide arrays in several different host strains was studied. These included the F, G1, G2 and U protospacers as well as a protospacer targeting the zwf promoter, Z. Strains that were evaluated included E. cloni 10G, a commercial recA1 cloning strain (Lucigen), as well as DLF_Z0025, a control host utilized for 2-stage dynamic metabolic control, lacking proteolytic degron tags on any metabolic enzymes, DLF_Z0045, with degron tags on GltA and UdhA, DLF_Z0047 (FGU, described above), as well as derivatives of DLF_Z0047 including a recA1 mutant (recAG160D), an sbcD gene deletion (a component of the SbcCD endonuclease recognizing hairpins and palindromic sequences present in guide arrays) and deletions in cas1 and cas2. Results are given in FIG. 1D.

Guide arrays were stable in the cloning strain. This result was not surprising as these constructs were originally constructed using E. cloni 10G and original plasmids confirmed via sequencing without any protospacer loss. Protospacer loss was first noticed in DLF_Z0025 for a small group of arrays. DLF_Z0025 has been modified for constitutive expression of the Cascade operon (FIG. 1A). Increased instability was detected with host strain DLF_Z0045 and DLF_Z0047. Neither incorporation of a recA1 mutation or a deletion of sbcD reduced protospacer loss in the DLF_Z0047 background. In the case of recA, this is consistent with previous studies demonstrating that although only 20 bp of homology will enable recombination in E. coli, homologous sequences greater than 50 bp are required for significant recombination; protospacers are 30 bp long. In contrast either a deletion in cas1 or cas2 improved array stability, with a cas1 deletion, having minimal protospacer loss.

The cas1 deletion results are consistent with the Cas1/2 endonuclease being responsible for protospacer loss, Cas1 being the nuclease component. This activity is consistent with their previously reported activity in protospacer acquisition. The fact that a very low-level of protospacer loss was still observed with a Cas1 mutation indicates the potential for a second alternative mechanism for protospacer loss, or alternatively inaccuracies in our PCR assay. However, as can be seen in FIG. 1D, guide arrays containing the F protospacer had noticeably more instability than those without. This protospacer specificity is not consistent with a generalized endonuclease activity, prompting us to further investigate while F containing arrays have an increased propensity for protospacer loss.

FabI may be a strictly essential enzyme, and despite the fact that the guide arrays are under inducible expression, leaky expression could lead to growth inhibition, and that guide arrays losing the F protospacer would have a selective advantage in strains where the Cascade operon (including cas1 and cas2) is overexpressed. This is also consistent with a general observation that transformation of guide array plasmids with an F protospacer results in lower colony numbers that other arrays. We constructed a plasmid (pFABI, FIG. 1E) enabling the expression of FabI from an alternative constitutive promoter, one which is not silenced by the F protospacer. We then assessed impact of cotransformation of pFABI with guide array plasmids containing F protospacers on colony numbers as well array stability. As can be seen in FIG. 1F-1G, cotransformation of pFABI increased colony numbers as well as array stability. These data are consistent with growth inhibition due to leaky silencing of fabI, and as a result a selective advantage of arrays where the F protospacer is lost.

Taken together, the results discussed above support a model wherein basal Cas1/2 endonuclease activity results in the loss of protospacers from guide arrays. Silencing arrays with protospacers targeting essential genes, may lead to growth inhibition, even if subtle, due to leaky expression of guides, when the Cascade operon is overexpressed. Arrays missing toxic protospacers can be amplified via selection in routine cultures. There are several options to improve array stability. Firstly, simply deleting cas1 should improve stability and as Cas1 is not required for the silencing function of the Cascade operon, gene silencing should not be affected. This approach would require two modifications to future silencing strains, the deletion of cas3 and cas1 (FIG. 1A). However, in light of the toxicity observed in case of basal fabI silencing, we also evaluated a second option, wherein we deleted cas3 and used a tightly controlled low phosphate inducible promoter to express the Cascade operon rather than a constitutive promoter (Biobrick J23100) as originally reported. To implement and test this approach we constructed a DLF_S0047, identical to DLF_Z0047, containing degron tags on FabI, GltA and UdhA, but wherein the constitutive J23100 promoter (FIG. 1A) was replaced by a tightly controlled low phosphate inducible modified yibD gene promoter, preceded by a strong synthetic transcriptional tZ terminator. Array instability was eliminated using DLF_S0047 as can be seen in FIG. 1D. Also constructed was DLF_S0025 as new stable strain for future engineering for dynamic metabolic control.

Utilization of Cascade for CRISPR interference will benefit from tighter control over Cascade operon (cas1/2) expression, if not deletion of cas1/2, or at least evaluation of guide stability.

### Disclosed Embodiments Are Non-Limiting

While various embodiments of the present invention have been shown and described herein, it is emphasized that such embodiments are provided by way of example only. Numerous variations, changes and substitutions may be made without departing from the invention herein in its various embodiments. Specifically, and for whatever reason, for any grouping of compounds, nucleic acid sequences, polypeptides including specific proteins including functional enzymes, metabolic pathway enzymes or intermediates, elements, or other compositions, or concentrations stated or otherwise presented herein in a list, table, or other grouping unless clearly stated otherwise, it is intended that each such grouping provides the basis for and serves to identify various subset embodiments, the subset embodiments in their broadest scope comprising every subset of such grouping by exclusion of one or more members (or subsets) of the respective stated grouping. Moreover, when any range is described herein, unless clearly stated otherwise, that range includes all values therein and all sub-ranges therein.

Also, and more generally, in accordance with disclosures, discussions, examples and embodiments herein, there may be employed conventional molecular biology, cellular biology, microbiology, and recombinant DNA techniques within the skill of the art. Such techniques are explained fully in the literature. See, e.g., Sambrook and Russell, “Molecular Cloning: A Laboratory Manual,” Third Edition 2001 (volumes 1-3), Cold Spring Harbor Laboratory Press, Cold Spring Harbor, N. Y.; Animal Cell Culture, R. I. Freshney, ed., 1986. These published resources are incorporated by reference herein.

The following published resources are incorporated by reference herein for description useful in conjunction with the invention described herein, for example, methods of industrial bio-production of chemical product(s) from sugar sources, and also industrial systems that may be used to achieve such conversion (Biochemical Engineering Fundamentals, 2^{nd} Ed. J. E. Bailey and D. F. Ollis, McGraw Hill, N.Y., 1986, e.g.Chapter 9, pages 533-657 for biological reactor design; Unit Operations of Chemical Engineering, 5^{th} Ed., W. L. McCabe et al., McGraw Hill, N.Y. 1993, e.g., for process and separation technologies analyses; Equilibrium Staged Separations, P. C. Wankat, Prentice Hall, Englewood Cliffs, N.J. USA, 1988, e.g., for separation technologies teachings).

All publications, patents, and patent applications mentioned in this specification are entirely incorporated by reference herein.

1. A genetically modified microorganism wherein:
an endogenous cas3 nuclease is deleted or mutated;
a Cascade operon operatively linked to a promotor that permits conditional overexpression of the operon; and
a guide array in which at least one CRISPR/Cascade gRNA may be conditionally expressed, wherein expression of the guide array results in reduced expression of at least one gene,
wherein the genetically modified microorganism is characterized by increased stability of the guide array when compared to a genetically modified microorganism lacking an endogenous cas3 nuclease deletion or mutation or conditional expression of a Cascade operon.

2. The genetically modified microorganism of claim 1, wherein an endogenous cas1 gene is deleted or mutated.

3. The genetically modified microorganism of claim 1, wherein the Cascade operon is overexpressed under the control of a tightly repressed inducible promoter.

4. The genetically modified microorganism of claim 3, wherein the tightly repressed inducible promoter is PhoB activated.

5. The genetically modified microorganism of claim 1, wherein the genetically modified microorganism is an E. coli microorganism.

6. The genetically modified microorganism of claim 1, wherein the reduced expression gene is selected from the group consisting of: fabI, gltA1, gltA2, udhA, and zwf.

7. A method of conditionally silencing a gene in a genetically modified microorganism, comprising
providing a genetically modified microorganism characterized by:
deletion or mutation of an endogenous cas3 nuclease;
a Cascade operon; and
at least one guide array comprising at least one CRISPR/Cascade gRNA,
growing the genetically modified microorganism under conditions wherein expression of the CRISPR/Cascade gRNA results in reduced expression of at least one gene of the genetically modified microorganism.

8. The method of claim 7 wherein the genetically modified microorganism further comprises deletion or mutation of an endogenous cas1 gene.

9. The method of claim 7 wherein the genetically modified microorganism is further characterized by the Cascade operon under the control of an inducible promoter that is a PhoB activated.

10. The method of claim 7, wherein the genetically modified microorganism is an E. coli microorganism.

11. The method of claim 7, wherein the reduced expression gene is selected from the group consisting of: fabI, gltA1, gltA2, udhA, and zwf.
